(19) United States
(12) Patent Application Publication
(10) Pub. No.: US 2021/0304001 A1
(43) Pub. Date: Sep. 30, 2021

(54) Multi-head neural network model to simultaneously predict multiple physiological signals from facial RGB video

(71) Applicant: Google LLC, Mountain View, CA (US)

(72) Inventors: Jack Po, Mountain View, CA (US); Jiening Zhan, Mountain View, CA (US); Jiang Wu, Mountain View, CA (US); Silviu Borac, Mountain View, CA (US); Yunus Emre, Mountain View, CA (US)

(21) Appl. No.: 17/215,915

(22) Filed: Mar. 29, 2021

Related U.S. Application Data
(60) Provisional application No. 63/001,639, filed on Mar. 30, 2020.

(51) Int. Cl. G06N 3/08; G06N 3/04; A61B 5/0205; G06K 9/00; G16H 40/67; G16H 80/00

(52) U.S. Cl. CPC G06N 3/08; G06N 3/04; A61B 5/0205; G06K 9/00315; G16H 40/67; G16H 80/00; G06K 9/00744

(57) ABSTRACT

A method for estimating two or more physiological signals from a subject includes steps of a) obtaining a video input in the form of a sequence of frames of image data depicting the face and optionally the chest of the subject; b) providing the video input to a multi-head neural network model trained from a set of facial video inputs from a multitude of other subjects (such video inputs optionally including the chest), wherein the model has at least two heads and is trained to predict at least two physiological signals from a video input; and c) generating with the model data representing an estimate of the two or more physiological signals of the subject. In one embodiment the physiological signals are heart rate and respiratory rate. In one embodiment the multi-head neural network model is implemented in a smartphone having a camera which is used to capture the video input.

### CROSS-REFERENCE TO RELATED APPLICATION

This application claims priority to U.S. Patent Application No. 63/001,639, filed Mar. 30, 2020, which is incorporated herein by reference.

### BACKGROUND

Sensors or other means can be used to measure physiological parameters of interest while in direct contact with the body. For example, blood pressure, heart rate, breathing rate, blood oxygenation, galvanic skin response, or other physiological parameters can be detected by detecting mechanical displacements, mechanical pressures, absorption or scattering spectra, electrical voltages, electrical impedances, or other physical properties of one or more parts of a body. Such physical properties can be detected using strain gauges, accelerometers, light emitters and detectors, electrodes, or other sensor means.

Alternatively, physiological parameters of interest can sometimes be measured in a non-contact manner, using cameras or other means that are remote from or otherwise not in contact with a part of the body. For example, video of a person's body could be used to determine breathing rate of the person. Some current implementations of video-derived vital sign parameters are based on signal processing methodologies, specifically by isolating the green channel of a RGB video feed, amplifying the signal, and then deriving a photoplethysmogram (PPG) from it. Once a PPG is derived, it is then used to derive the heart rate/respiratory rate based on existing, well-known algorithms. Sometimes, optical flow methods are used to detect respiratory rate separate from the method above.

Background art includes U.S. patent application publications 2019/0117151; U.S. 2018/0289334; and US 2017/0367590; Fan et al., Multi-region ensemble convolutional neural network for facial expression recognition, arXiv:1807.10575 [cs.CV] (2018); and Yin et al. A multi-modal hierarchical recurrent neural network for depression detection, AVEC '19: Proceedings of the 9th International on Audio/Visual Emotion Challenge and Workshop October 2019 Pages 65-71.

### SUMMARY

This disclosure relates to a method and system for generating data representing estimates of multiple physiological signals, such as heart rate and respiratory rate, from an input in the form of RGB video frames of the face of a subject, e.g., captured by a smartphone camera. In our method we use a multi-head neural network model to generate such data.

The term “multi-head neural network model” is used to refer to a machine learning model in the form of trained neural network that has more than one output layers and associated predictions (referred to as “heads”). For example, a neural network that is trained to make two different predictions from a single input (a sequence of RGB video frames of the face of a subject in this instance), has two different output layers and associated predictions, and can be said to have two heads. In our multi-head neural network model described in this document most of the network weights will remain shared across the heads. This enables the network to learn features that are helpful for multiple predictions.

While the following disclosure gives an example of two-head neural network model predicting heart rate and respiratory rate, with sufficient training examples the model could be developed with additional output layers and associated predictions to make a third or even fourth prediction of still further physiological parameters based on an input facial video sequence. Therefore, the present description is offered by way of example and not limitation. In some examples the portions of a trained model corresponding specifically to the third (fourth, etc.) physiological parameter could be trained at the same time that common portion (e.g., CNN parameters) and the portions corresponding specifically to the first and second physiological parameters. Alternatively, the portions corresponding specifically to the third (fourth, etc.) physiological parameter could be trained separately from the other portions of the model, e.g., to allow the additional portions to be added later (e.g., as additional downloaded model components). In such an example, training the portions corresponding specifically to the third (fourth, etc.) physiological parameter could include using the common portions to generate intermediate outputs/inputs in order to update the portions corresponding specifically to the third (fourth, etc.) physiological parameter while not updating or otherwise changing the common portions (e.g., such that the portions specific to the first and second physiological parameters are still able to rely on the intermediate outputs/input from the common portions in order to produce accurate predictions of the first and second physiological parameters).

In this disclosure end-to-end neural network models are provided that receive as input facial videos and that generate as output physiological parameters, thus eliminating much of the extensive and costly tuning required for conventional signal processing based methodologies.

The set of input facial videos used to train the models described herein are preferentially selected to represent a wide range of facial characteristics, representing individuals spanning the space of human facial characteristic variability. Similarly, facial videos used to validate such models (e.g., to ensure that the models have not been over-fitted to the training data, to verify a degree of accuracy or other statistics for the model predictions) are preferentially selected to represent a wide range of facial characteristics. This selection of widely representative training and validation data is done to ensure that the resulting models provide accurate predictions for as wide a range of potential users as possible. Additionally, this more varied training data can result in trained models that are more robust and that can provide accurate predictions across a wider range of use conditions.

In this document, when we refer to videos of the “face” we mean that expression to include video which captures at least the face of the subject. In practice such videos could include other areas such as the neck, upper chest, etc. In some applications, it may be desirable to include in the facial video the chest, that is, the middle and upper portion of the torso, as chest video may provide a better signal from some physiological parameters, such as respiratory rate.

In one aspect of this disclosure, a method for estimating two or more physiological signals from a subject is disclosed. The method includes steps of: a) obtaining a video input in the form of a sequence of frames of image data depicting the face of the subject; b) providing the video input to a multi-head neural network model trained from a set of facial video inputs from a multitude of other subjects, wherein the model has at least two heads and is trained to predict at least two physiological signals from a video input; and c) generating with the model data representing an estimate of the two or more physiological signals of the subject. In one embodiment, the video also includes the chest of the subject. In this embodiment the physiological signals are heart rate and respiratory rate. In one embodiment the multi-head neural network model is implemented in a smartphone having a camera which is used to capture the video input. Alternatively, the model is implemented in a computing resource remote from the smartphone.

In another aspect, apparatus for estimating two or more physiological signals from a subject is described which includes a smartphone having a camera obtaining a video input in the form of a sequence of frames of image data depicting the face of the subject; and a multi-head neural network model trained from a set of facial video inputs from a multitude of other subjects, wherein the model is trained to predict at least two physiological signals. The model is configured to receive the video input and generate data representing an estimate of the two or more physiological signals of the subject.

In another aspect a system includes a controller and a computer-readable medium having stored thereon program instructions that, upon execution by the controller, cause the controller to perform the any of the above methods. Such a computer-readable medium can be non-transitory.

In another aspect a system includes a computer-readable medium having stored thereon program instructions that, upon execution by the controller, cause the controller to perform the any of the above methods. Such a computer-readable medium can be non-transitory and can be incorporated into an article of manufacture.

These as well as other aspects, advantages, and alternatives will become apparent to those of ordinary skill in the art by reading the following detailed description with reference where appropriate to the accompanying drawings. Further, it should be understood that the description provided in this summary section and elsewhere in this document is intended to illustrate the claimed subject matter by way of example and not by way of limitation.

### BRIEF DESCRIPTION OF THE FIGURES

FIG. 1 is an illustration of multi-head neural network which includes two heads for prediction of heart rate and respiratory rate from an input sequence of RGB video frames capturing images of the face of a subject.

FIG. 2 is a diagram showing the training of the neural network of FIG. 1 and its use as a trained model to make predictions from an input video sequence.

FIG. 3 is a diagram showing a series of screen shots on a smartphone having a camera showing the manner of use of the trained model of FIG. 2 to make two or more physiological predictions. Such predictions can be generated and reported locally on the smartphone or transmitted over wireless and computer networks to remote computing resources. Alternatively, the computation of the physiological parameters could be performed in a remote computing resource as shown in FIG. 3.

FIG. 4 is a simplified block diagram showing some of the components of an example computing system.

FIG. 5 is a flowchart of a method.

### DETAILED DESCRIPTION

Examples of methods and systems are described herein. It should be understood that the words “exemplary,” “example,” and “illustrative,” are used herein to mean “serving as an example, instance, or illustration.” Any embodiment or feature described herein as “exemplary,” “example,” or “illustrative,” is not necessarily to be construed as preferred or advantageous over other embodiments or features. Further, the exemplary embodiments described herein are not meant to be limiting. It will be readily understood that certain aspects of the disclosed systems and methods can be arranged and combined in a wide variety of different configurations.

### I. Overview

This document describes a method for estimating two or more physiological signals, e.g., heart rate and respiratory rate, from a subject. The method of this disclosure aims to enhance mobile healthcare (e.g. telehealth, global access to care) by developing technology that can provide mobile diagnostics via a consumer-grade smartphone equipped with a camera. Smartphones are ubiquitous, and as care moves away from clinics and hospitals, they can be used to provide objective health data for the users and for the care providers. Smartphone cameras in particular, are becoming increasingly higher quality, and can be used as a health sensor and data acquisition device.

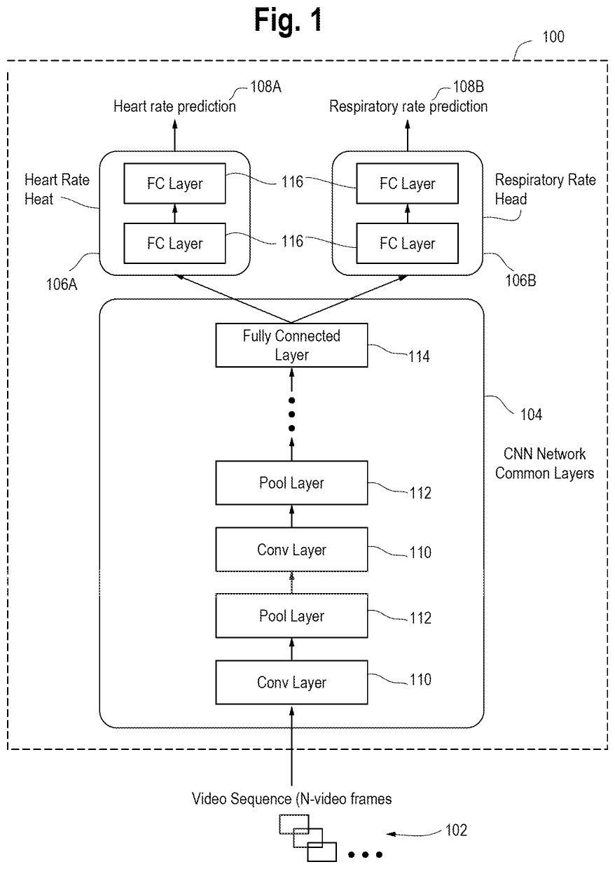

In particular, and referring now to FIG. 1, in our method we obtain a video input 102 in the form of a sequence of N frames of image data depicting the face of the subject. This can be simply the acquisition of a color (RGB) video of the face of the subject for a duration of example 10, 20 or 30 seconds at a frame rate of consumer grade cameras incorporated in smartphones, for example 20 or 30 frames per second. The video input is provided to a multi-head neural network model 100 trained from a set of facial video inputs from a multitude of other subjects. The model 100 includes two heads 106A, 106B which generate prediction outputs 108A, 108B, such as data representing an estimate of heart rate and respiratory rate, respectively, of the subject. Thus, the model 100 is trained to predict at least two physiological signals from the video input 102 and makes such predictions at the time of use.

FIG. 1 is an illustration of one possible configuration of the multi-head neural network model 100. Before describing it in detail, a few general comments are offered first. In many machine learning problems with multiple objectives (i.e. with the same input data, one would like to derive and optimize multiple signals at once), a particular type of neural net model is used, namely a multi-head neural net model. This is the architecture that we have used for our method and system and shown in FIG. 1.

The multi-head neural network of FIG. 1 has several features:

1) it allows the same input data 102 (in the present circumstances, a sequence of N RGB video image frames) to be used for multiple prediction outcomes 108A, 108B at the same time;

2) it allows training of one model 100 that can be used for multiple outcomes at the same time, thus creating a model that is more robust to noise and features, or in other words provides for implicit data augmentation. Soft parameter sharing is used in the current model architecture.

3) it is easier to deploy in production, e.g. in processing units of a smartphone, since instead of running multiple independent machine learning models (one for each physiological signal), only one model 100 is needed and much of the computation work comes from the same pipeline, except for the last few output-specific layers (i.e., prediction layers).

In FIG. 1, the multi-head neural network model 100 includes a deep convolutional neural network 104 having stacked convolution layers 110 and pool layers 112, and a fully connected layer 114. These layers 110, 112, 114 are shown as CNN common layers 104, in other words, these layers are shared or are common to the two predictions generated by the model. Parameter sharing for the two predictions occurs within the convolutional and pool layers 110, 112.

The input of the fully connected layer 114 is the flattened output of the CNN and pooling layers 110 and 112. The output of the fully connected layer 114 is provided to two heads, one (106A) generating a prediction of heart rate (108A) and the other head (106B) generating a prediction of respiratory rate (108B). The two heads each include two stacked fully connected layers 116.

Regarding the design of the CNN convolutional neural network 104, this can take the form of a deep CNN known as Inception and described in the article of C. Szegedy et al. “Going deeper with convolutions” arXiv.org 1409.4842v1 [cs.CV] (17 Sep. 2014). The Szegedy et al. article discloses a single head neural network used for image classification. The Szegedy et al. article is incorporated by reference herein. The article describes a suitable neural network architecture that can be trained to generate estimates of physiological signals, such as heart rate or respiratory rate from frames of imagery, as in the present example. For a multi-head version of this architecture, there are multiple output fully connected layers and predictions (heads), as shown in FIG. 1. Most of the network weights will remain shared across the heads 106A, 106B. This enables the network to learn features that are helpful for multiple predictions.

Other examples of deep convolutional neural networks that could be used, albeit with modification to add a second prediction head, are disclosed in C. Szegedy et al., Rethinking the Inception Architecture for Computer Vision, arXiv:1512.00567 [cs.CV] (December 2015); see also US patent application of C. Szegedy et al., “Processing Images Using Deep Neural Networks”, Ser. No. 14/839,452 filed Aug. 28, 2015. A fourth generation, known as Inception-v4 is considered an alternative architecture. See C. Szegedy et al., Inception-v4, Inception-ResNet and the Impact of Residual Connections on Learning, arXiv:1602.0761 [cs.CV] (February 2016). See also US patent application of C. Vanhoucke, “Image Classification Neural Networks”, Ser. No. 15/395,530 filed Dec. 30, 2016.

It will be noted that the predictions of FIG. 1 are rate predictions, and not merely image classifications. The rate predictions are based on subsets of the input frames. In implementing the model 100 of FIG. 1, we assume that the input 102 is 30 frames per second (fps) although we can upsample/downsample the input if the capture fps is different. Additionally or alternatively, the input 102 could be provided to the model 100 without resampling and the output 108A/B of the model 100 then scaled according to the fps of the input 102 in order to provide an accurate estimate of heart rate/respiratory rate, or of some other estimated output variable that is related to time and thus which would be increased or decreased as the frame rate of the input 102 is increased/decreased. The input 102 to the model is a sequence of N frames. Depending on the desired output frequency (i.e., how often rate is predicted and reported), we can use a sliding window, e.g. if we would like to output every second and use 2 seconds of frames (60 frames at 30 fps) to make the prediction, we use a window of 2 seconds which slides at 1 second.

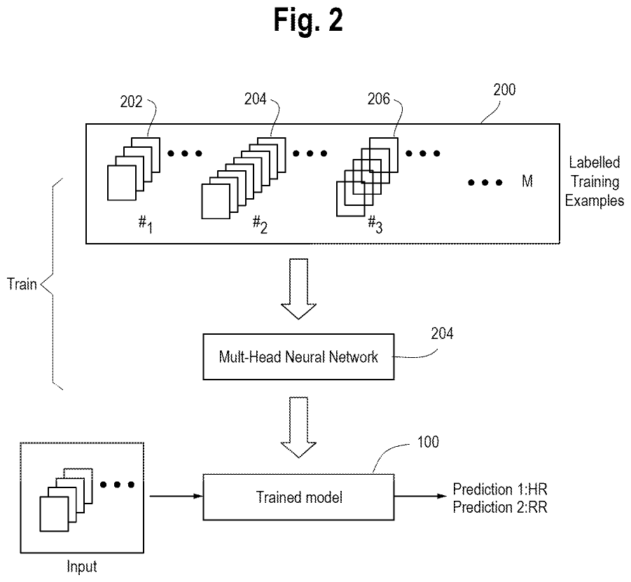

FIG. 2 shows the training of the neural network model of FIG. 1 and its use as a trained model to make predictions from an input video sequence. In particular, for training we first obtain a set of labeled training examples or data 200. This data is in the form of a sequence of video frames of faces from a multitude of individuals. For example, for person #1 we have a 10 second RGB video sequence 202 at 30 fps, for person #2 we have a 25 second RGB video sequence at 60 fps, for person #3 we have a 30 second RGB video sequence at 30 fps, etc, for M subjects, where M is preferably >100 and more preferably >1,000. These training data 200 are input to a multi-head neural network 204, essentially of the design of FIG. 1, and in a training exercise this network learns the parameters and weights needed to make accurate predictions of heart rate and respiratory rate. The result of this training exercise is a trained multi-head neural network model 100 of FIG. 1. This model then can be used to make a prediction of heart rate and respiratory rate for a new, previously unseen video input 102 as shown in FIGS. 1 and 2.

Example Application/Use Cases

We contemplate a use case in which the multi-head neural network can be implemented in a mobile diagnostics scenario. In particular, we aim to enhance mobile healthcare (e.g. telehealth, global access to care) by developing technology that can provide mobile diagnostics via a consumer-grade smartphone. Smartphones are ubiquitous, as care moves away from clinics and hospitals, they can be used to provide objective health data for the users and for the care providers. Smartphone cameras in particular, are becoming increasingly higher quality, and can be used as a health sensor.

Remote monitoring (active case) is another use case. What we mean by “active” is that a patient has to actively go to a device in order for measurements to happen. The smart display evaluates some parameters for wellness reasons (e.g., heart rate) when a person is looking at the display. In one embodiment, this is implemented on a mobile device.

In another possible embodiment, this is implemented in the processing unit of the smart display, which includes code and parameters implementing the trained model 100 of FIGS. 1 and 2 and generates the predictions and reports them in substantial real time.

Other embodiments include other types of cameras, or displays, having their own processing power or in communication with remote processing units.

Several additional specific applications or use cases are contemplated:

1) The use of the technology on a smartphone by a potential caregiver/patient. In this embodiment, the smartphone includes both the camera functionality to capture the input video frames as well as the computing resources to execute the trained model 100 of FIGS. 1 and 2 to generate outputs in the form of estimated heart rate and respiratory rate in real time. For example, a parent has a sick baby, and they call their pediatrician. The pediatrician informs the parent that they would like to know the heart rate and respiratory rate of the child to figure out whether the child might be having an asthma attack. The parent captures a 20 second video of the child's face with the smartphone camera and the video is fed as input to the trained model 100 of FIGS. 1 and 2. (An app on the smartphone with suitable user prompts can facilitate this interaction). The model generates the predictions of heart rate and respiratory rate and the parent provides this information verbally to the pediatrician to help them assess the possibility of an asthma attack. Alternatively, the app could be configured with tools or software to transmit the heart rate and respiratory rate estimates securely transmitted to a portal that can store medical information and facilitate communication between providers and patients.

2) The use of the technology on a smartphone/computer by a physician in a telemedicine scenario with a remote party. This remote party might either be a patient, or another provider. As an example, a cardiologist might be called by a nurse in a nursing home for a remote consult for an elderly patient. The cardiologist would want certain physiological parameters from the patient they are consulting on remotely. The nurse then captures a 15 second video of the face of the patient. The video could be processed by the trained model either locally on the nurse's smartphone (or local computing resource, such as nurse's station in the nursing home) and immediate generate the predictions of heart rate and respiratory rate, which are provided to the cardiologist over the telephone. Or, alternatively, the video could be transmitted over cellular and computer networks to the cardiologist where there is a computing resource the implements the trained model of FIG. 2, and the computing resource provides the estimates of heart rate and respiratory rate to the cardiologist to facilitate the consult.

Another use case example would be what most patients think of as a telemedicine call. A patient has a severe cough and makes a telephone call to a telemedicine provider to seek care using their smartphone. The provider/doctor might want to know more about the patient's physiological parameters (i.e. heart rate/respiratory rate) in order to make a better clinical decision. So, the patient is prompted to capture a 20 second video image of their face and chest with their smartphone camera. Computing resources on the smartphone execute the trained model of FIG. 2 on the input video sequence and generate the predictions of heart rate and respiratory rate and that information is provide to the provider/doctor to facilitate clinical decision making. For example, the rate information is presented on the display of the smartphone and the patient reads it to the telemedicine provider during the telephone call.

Note that this example illustrates several of the benefits of the embodiments described herein. By using many elements of the trained model in common between two (or more) predicted outputs, the model can occupy less storage on a device and require less memory and compute power. Accordingly, the model can be stored and computed on a user's smartphone or other limited-resource system local to a user (e.g., a tablet, a laptop, etc.). This allows the user to receive the benefits of the method without sending the input video to a remote server or other remote computing resource. This protects the user's privacy by avoiding sending video data of the user over a communications channel (e.g., the internet). This also reduces the bandwidth needed to perform the method, as the video data is used locally instead of being sent to some remote computing resource.

As another use case scenario, the methods of this disclosure could be practiced as part of a patient check-in process (i.e. at a check-in kiosk or desk), e.g., at a doctor's office, hospital or clinic in which the patient's vital signs are measured at intake. The staff or personnel at the check-in kiosk (e.g., receptionist) will have the smartphone with camera and built-in trained multi-head neural network to make physiological predictions. The personnel at the kiosk can thus get some vital signs for the patient at the same time as the patient checks in for their appointment. Of course, the camera could also be an accessory to a desk-top computer and the computer contains the processing unit and memory for implementing the trained multi-head neural network and makes the physiological predictions. In another possible configuration, the check-in kiosk could have the smart display with camera as described earlier.

From the above discussion, it will be apparent that the trained multi-head neural network model could be implemented in a smartphone and/or run on a remote computing platform, e.g., in a computer at a doctor's office or clinic, or in a cloud server. This is similar to the way that speech recognition models are now implemented for mobile devices.

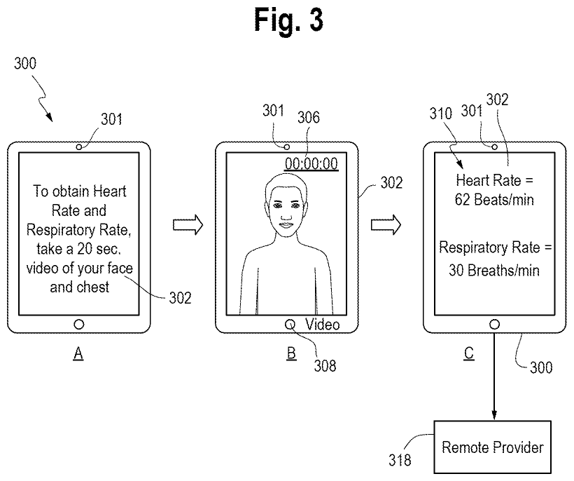

FIG. 3 is a diagram showing a series of screen shots on a smartphone 300 having a camera 301 showing the manner of use of the trained model of FIG. 2 to make two or more physiological predictions. For example, FIG. 3 could be considered to show the steps that an app resident on the smartphone 300 could execute to facilitate capture of the facial video. Once the app is located on the device and initiated, in Step A the screen 302 displays a prompt for the user: “To obtain heart rate and respiratory rate, take a 20 second video of your face.” The screen then reverts to that shown in Step B, in which the camera image is shown on the display 302 (the face including chest of the user) along with a timer 306 and a video initiate button or icon 308. When the button 308 is pressed the camera 301 starts collecting and storing the video image frames and timer 306 starts to run. After 20 seconds has elapsed, the app reverts to Step C and the display 302 shows the prediction 310 of the patient's heart rate and respiratory rate. Optionally, the smartphone transmits the heart and respiratory rate information to a remotely located healthcare provider 318, e.g., their doctor. In this scenario, the model 100 of FIGS. 1 and 2 is stored and run locally in a processing unit of the smartphone. As an alternative, the video frames captured in Step B are sent over a network to a secure remote computing resource in the cloud which stores and executes the trained model 100 and the determination of heart rate and respiratory rate predictions is done in the remote processing unit. The results could be sent back via the network to the smartphone 300 or provided to remotely located providers 318 as shown in FIG. 3.

The illustration of FIG. 3 is offered only by way of example and not limitation and of course the details of the user interface, prompts, and network configuration are offered only by way of example and not limitation. While the present disclosure has featured certain types of devices, such as smartphones and smart displays, in which the methods can be practiced it will be appreciated that other types of computing devices could be used instead, such as for example laptop computers, desktop computers, tablets, and still others, and all such types of computing devices are within the scope of this disclosure. Furthermore, while wireless transmission is one possible method for transmission of video data or predictions of physiological signals, or predicted rates and such, other types of transmission formats are of course possible and within the scope of this disclosure. All questions as to the scope of the present disclosure is to be answered by reference to the appended claims.

### II. Illustrative Systems

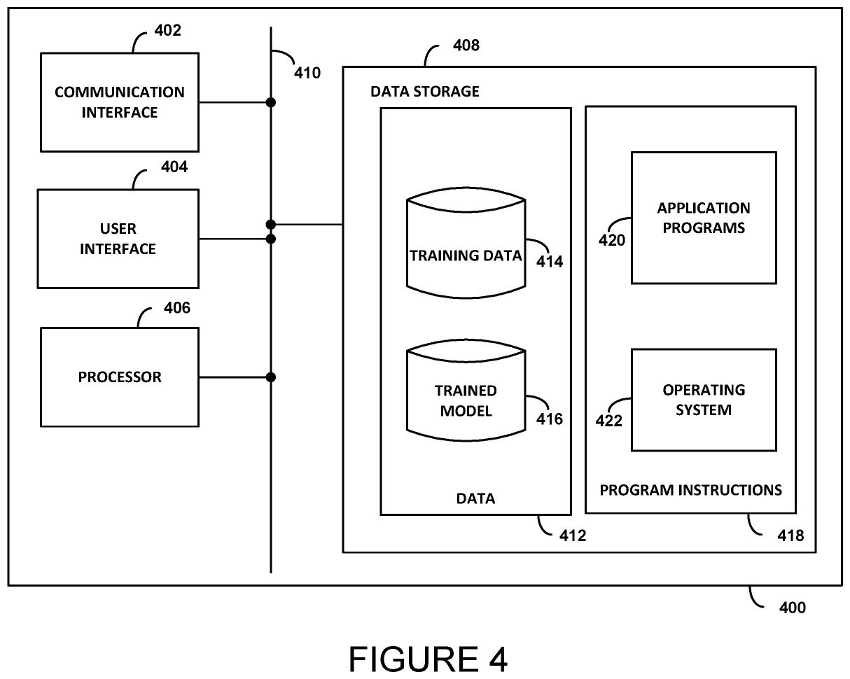

FIG. 4 illustrates an example computing device 400 that may be used to implement the methods described herein. By way of example and without limitation, computing device 400 may be a cellular mobile telephone (e.g., a smartphone), a video camera, a computer (such as a desktop, notebook, tablet, or handheld computer), a personal digital assistant (PDA), a home automation component, a digital video recorder (DVR), a digital television, a remote control, a wearable computing device, a robot, a drone, an autonomous vehicle, or some other type of device. Such a device may be equipped with an image capture device so as to generate video (e.g., frames of a video stream at a specified frame rate) that may then be used as input to a model as described herein to produce estimates of two (or more) physiological parameters (e.g., heart rate and breathing rate) for a person depicted in the video. It should be understood that computing device 400 may represent a physical camera device such as a digital camera, a particular physical hardware platform on which an image processing application operates in software, or other combinations of hardware and software that are configured to carry out image processing and/or model evaluation functions.

As shown in FIG. 4, computing device 400 may include a communication interface 402, a user interface 404, a processor 406, and data storage 408, all of which may be communicatively linked together by a system bus, network, or other connection mechanism 410.

Communication interface 402 may function to allow computing device 400 to communicate, using analog or digital modulation of electric, magnetic, electromagnetic, optical, or other signals, with other devices, access networks, and/or transport networks. Thus, communication interface 402 may facilitate circuit-switched and/or packet-switched communication, such as plain old telephone service (POTS) communication and/or Internet protocol (IP) or other packetized communication. For instance, communication interface 402 may include a chipset and antenna arranged for wireless communication with a radio access network or an access point. Also, communication interface 402 may take the form of or include a wireline interface, such as an Ethernet, Universal Serial Bus (USB), or High-Definition Multimedia Interface (HDMI) port. Communication interface 402 may also take the form of or include a wireless interface, such as a Wifi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 402. Furthermore, communication interface 402 may comprise multiple physical communication interfaces (e.g., a Wifi interface, a BLUETOOTH® interface, and a wide-area wireless interface).

In some embodiments, communication interface 402 may function to allow computing device 400 to communicate, with other devices, remote servers, access networks, and/or transport networks. For example, the communication interface 402 may function to access a trained model via communication with a remote server or other remote device or system in order to allow the computing device 400 to use the trained model to predict, based on frames of an RGB video, multiple physiological parameters of a person whose face or other body part(s) are represented in the video. For example, the computing system 400 could be a cell phone, digital camera, or other image capturing device and the remote system could be a server containing a memory containing such a trained model.

User interface 404 may function to allow computing device 400 to interact with a user, for example to receive input from and/or to provide output to the user. Thus, user interface 404 may include input components such as a keypad, keyboard, touch-sensitive or presence-sensitive panel, computer mouse, trackball, joystick, microphone, and so on. User interface 404 may also include one or more output components such as a display screen which, for example, may be combined with a presence-sensitive panel. The display screen may be based on CRT, LCD, and/or LED technologies, or other technologies now known or later developed. User interface 404 may also be configured to generate audible output(s), via a speaker, speaker jack, audio output port, audio output device, earphones, and/or other similar devices.

Processor 406 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). In some instances, special purpose processors may be capable of image processing, image alignment, merging images, evaluating neural network models or other machine learning models, among other applications or functions. Data storage 408 may include one or more volatile and/or non-volatile storage components, such as magnetic, optical, flash, or organic storage, and may be integrated in whole or in part with processor 406. Data storage 408 may include removable and/or non-removable components.

Processor 406 may be capable of executing program instructions 418 (e.g., compiled or non-compiled program logic and/or machine code) stored in data storage 408 to carry out the various functions described herein. Therefore, data storage 408 may include a non-transitory computer-readable medium, having stored thereon program instructions that, upon execution by computing device 400, cause computing device 400 to carry out any of the methods, processes, or functions disclosed in this specification and/or the accompanying drawings. The execution of program instructions 418 by processor 406 may result in processor 406 using data 412.

By way of example, program instructions 418 may include an operating system 422 (e.g., an operating system kernel, device driver(s), and/or other modules) and one or more application programs 420 (e.g., camera functions, model and/or ANN training, RGB video-based multiple parameter estimation) installed on computing device 400. Data 412 may include training videos and associated physiological parameter values 414 and/or one or more trained models 416. Training data 414 may be used to train a multi-headed model as described herein (e.g., to generate and/or update the trained model 416). The trained model 416 may be applied to generate estimated heart rates, breathing rates, or other physiological parameter values based on input video clips (e.g., frames of video captured using camera components of the device 400 and/or accessed via the communication interface 402).

Application programs 420 may communicate with operating system 422 through one or more application programming interfaces (APIs). These APIs may facilitate, for instance, application programs 420 reading and/or writing a trained model 416, transmitting or receiving information via communication interface 402, receiving and/or displaying information on user interface 404, capturing video using camera components 424, and so on.

Application programs 420 may take the form of “apps” that could be downloadable to computing device 400 through one or more online application stores or application markets (via, e.g., the communication interface 402). However, application programs can also be installed on computing device 400 in other ways, such as via a web browser or through a physical interface (e.g., a USB port) of the computing device 400.

### III. Example Methods

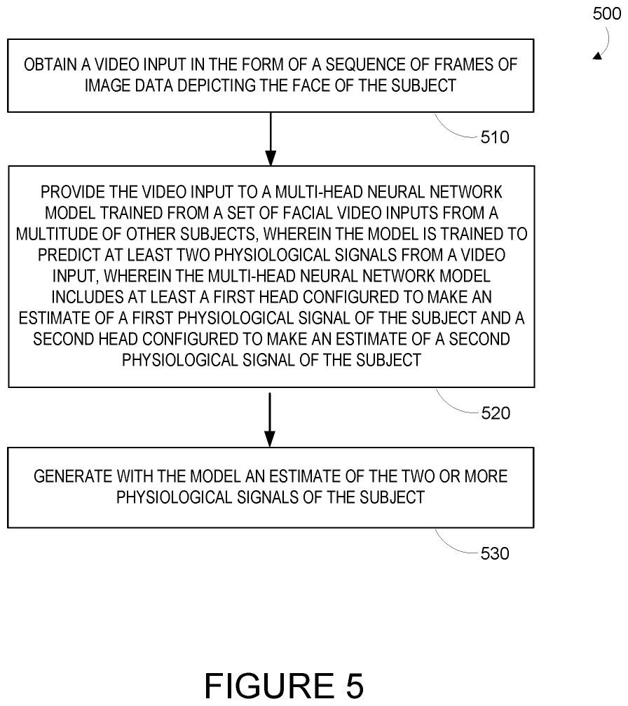

FIG. 5 is a flowchart of a method 500 for estimating two or more physiological signals of a subject. The method 500 includes obtaining a video input in the form of a sequence of frames of image data depicting the face of the subject (510). The method 500 additionally includes providing the video input to a multi-head neural network model trained from a set of facial video inputs from a multitude of other subjects, wherein the model is trained to predict at least two physiological signals from a video input, wherein the multi-head neural network model includes at least a first head configured to make an estimate of a first physiological signal of the subject and a second head configured to make an estimate of a second physiological signal of the subject (520). The method 500 additionally includes generating with the model an estimate of the two or more physiological signals of the subject (530). The method 500 could include additional or alternative elements or features.

### IV. Conclusion

The above detailed description describes various features and functions of the disclosed systems, devices, and methods with reference to the accompanying figures. In the figures, similar symbols typically identify similar components, unless the context indicates otherwise. The illustrative embodiments described in the detailed description, figures, and claims are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the message flow diagrams, scenarios, and flowcharts in the figures and as discussed herein, each step, block and/or communication may represent a processing of information and/or a transmission of information in accordance with example embodiments. Alternative embodiments are included within the scope of these example embodiments. In these alternative embodiments, for example, functions described as steps, blocks, transmissions, communications, requests, responses, and/or messages may be executed out of order from that shown or discussed, including in substantially concurrent or in reverse order, depending on the functionality involved. Further, more or fewer steps, blocks and/or functions may be used with any of the message flow diagrams, scenarios, and flow charts discussed herein, and these message flow diagrams, scenarios, and flow charts may be combined with one another, in part or in whole.

A step or block that represents a processing of information may correspond to circuitry that can be configured to perform the specific logical functions of a herein-described method or technique. Alternatively or additionally, a step or block that represents a processing of information may correspond to a module, a segment, or a portion of program code (including related data). The program code may include one or more instructions executable by a processor for implementing specific logical functions or actions in the method or technique. The program code and/or related data may be stored on any type of computer-readable medium, such as a storage device, including a disk drive, a hard drive, or other storage media.

The computer-readable medium may also include non-transitory computer-readable media such as computer-readable media that stores data for short periods of time like register memory, processor cache, and/or random access memory (RAM). The computer-readable media may also include non-transitory computer-readable media that stores program code and/or data for longer periods of time, such as secondary or persistent long term storage, like read only memory (ROM), optical or magnetic disks, and/or compact-disc read only memory (CD-ROM), for example. The computer-readable media may also be any other volatile or non-volatile storage systems. A computer-readable medium may be considered a computer-readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

We claim:

1. A method for estimating two or more physiological signals of a subject, comprising the steps of:
obtaining a video input in the form of a sequence of frames of image data depicting the face of the subject;
providing the video input to a multi-head neural network model trained from a set of facial video inputs from a multitude of other subjects, wherein the model is trained to predict at least two physiological signals from a video input, wherein the multi-head neural network model includes at least a first head configured to make an estimate of a first physiological signal of the subject and a second head configured to make an estimate of a second physiological signal of the subject;
generating with the model an estimate of the two or more physiological signals of the subject.

2. The method of claim 1, wherein the sequence of frames of image data depict the face and the chest of the subject and wherein the first and second physiological signals comprise heart rate and respiratory rate, respectively.

3. The method of claim 1, wherein obtaining the video input comprises operating a camera of a smartphone to generate the sequence of frames of image data and wherein providing the video input to the multi-head neural network model and generating with the model the estimate of the two or more physiological signals are performed by one or more processors of the smartphone.

4. The method of claim 1, further comprising:
transmitting, by the smartphone over a communications network, an indication of the generated estimate of the two or more physiological signals of the subject.

5. The method of claim 1, wherein obtaining the video input comprises operating a camera of a smartphone to generate the sequence of frames of image data.

6. The method of claim 5, further comprising:
transmitting an indication of the video input from the smartphone to a remote computing resource, wherein providing the video input to the multi-head neural network model and generating with the model the estimate of the two or more physiological signals are performed by the remote computing resource.

7. The method of claim 1, wherein the method is implemented in one or more computing resources facilitating check-in at a medical office, clinic or hospital.

8. The method of claim 7, wherein the one or more computing resources comprises a smartphone.

9. The method of claim 1, wherein the method is executed in a smart display.

10. The method of claim 1, wherein the multi-head neural network model includes a common portion that generates an intermediate output that is received, as an input, by the first head and the second head to generate the estimates of the first and second physiological signals of the subject, respectively, and wherein the method further comprises:
using the set of facial video inputs from the multitude of other subjects to train the multi-head neural network model to predict the first and second physiological signals, wherein training the multi-head neural network model to predict the first and second physiological signals comprises updating parameters of the common portion, the first head, and the second head of the multi-head neural network model a plurality of times; and
using an additional set of facial video inputs, training a third head of the multi-head neural network model to predict a third physiological signal without altering the parameters of the common portion of the multi-head neural network model.

11. A smartphone configured for estimating two or more physiological signals of a subject, the smartphone comprising:
a camera; and
a controller comprising one or more processors, wherein the controller is configured to perform controller operations comprising:
operating the camera to obtain a video input in the form of a sequence of frames of image data depicting the face of the subject; and
providing the video input to a multi-head neural network model trained from a set of facial video inputs from a multitude of other subjects, wherein the model is trained to predict at least two physiological signals from a video input, wherein the multi-head neural network model includes at least a first head configured to make an estimate of a first physiological signal of the subject and a second head configured to make an estimate of a second physiological signal of the subject; and
generating with the model an estimate of the two or more physiological signals of the subject.

12. The smartphone of claim 11, wherein a representation of the multi-head neural network model is stored in a memory of the smartphone.

13. The smartphone of claim 11, wherein the first and second physiological signals comprise heart rate and respiratory rate, respectively.

14. The smartphone of claim 11, wherein the controller operations further comprise:
transmitting, by the smartphone over a communications network, an indication of the generated estimate of the two or more physiological signals of the subject.

15. The smartphone of claim 11, wherein the video input comprises a sequence of frames of RGB color images.

16. A method of remotely monitoring physiological parameters of a patient, comprising the steps of:
obtaining a video of the face of the subject over a communications network;
providing the video of the face of the subject to a multi-head neural network model trained from a set of facial video inputs from a multitude of other subjects, wherein the model is trained to predict at least two physiological parameters from a video input, wherein the multi-head neural network model includes at least a first head configured to make an estimate of a first physiological parameter of the subject and a second head configured to make an estimate of a second physiological parameter of the subject, and
generating with the model an estimate of the physiological parameters of the patient.

17. The method of claim 16, wherein the video input comprises a sequence of frames of RGB color images obtained from a smartphone.

18. The method of claim 17, further comprising the step of reporting the estimate of the physiological parameters to a remotely located medical provider.

19. The method of claim 17, further comprising the step of reporting the estimate of the physiological parameters to a communications device providing the video.

20. The method of claim 16, wherein the first and second physiological signals comprise heart rate and respiratory rate, respectively.
